(19) United States
(12) Patent Application Publication
(10) Pub. No.: US 2023/0025682 A1
(43) Pub. Date: Jan. 26, 2023

(54) ONSITE MICROORGANISM-BASED TREATMENT METHOD, SYSTEM AND APPARATUS

(71) Applicant: Robert GREEN, Pokeno Auckland (NZ)

(72) Inventor: Robert GREEN, Pokeno Auckland (NZ)

(21) Appl. No.: 17/781,885

(22) PCT Filed: Dec. 2, 2020
(86) PCT No.: PCT/NZ2020/050164

§ 371 Date: Jun. 2, 2022

(30) Foreign Application Priority Data
Dec. 3, 2019 (NZ) 759816

(51) Int. Cl. C02F 3/34; C12M 1/00; C12N 1/20

(52) U.S. Cl. CPC C02F 3/343; C12M 1/005; C12M 23/34; C12N 1/205; C02F 2305/04; C02F 2305/06; C02F 2307/08; C02F 3/348; C12M 1/04

(57) ABSTRACT

Conventional methods for treating fat, oil, grease (FOG) and other build-up in wastewater systems (including grease traps) of restaurants and the like typically rely on chemical-based detergents, which may be damaging both to the environment and to the wastewater system itself. While some bio-friendly alternatives are known, a common problem with all these agents is that they are “flushed through” the system rapidly, and thus are relatively ineffective and inefficient. In providing an onsite system and method comprising cultivating micro-organisms and then using a carrier to deliver them to an affected environment, the present invention provides a solution that is more efficient in requiring less “starter” ingredients as well as more effective in ensuring the cultivated micro-organisms are delivered to, maintain sustained contact with, and have adequate time to treat, the undesirable substance(s).

### FIELD OF THE INVENTION

The present invention relates to an onsite method, system and apparatus for treating an undesirable substance in an affected environment using one or more types of micro-organism. The invention has particular application to treating food-related build-up or residue (fat, oil, grease, and/or biofilms) in wastewater systems of restaurants and the like. However, the invention may also have a range of other applications.

### BACKGROUND OF INVENTION

In commercial food-preparation settings such as restaurants, disposal of food waste into the wastewater system can cause significant and expensive complications. Food tends to be relatively rich in fat/oil/grease (“FOG”) and/or sugar, among other things. When disposed of into wastewater systems, these substances (and/or their by-products) tend to form a build-up, or residue, on the inside of drains, pipes or other components, eventually obstructing them. Local government bodies tend to impose penalties on establishments that do not take adequate measures to prevent this from happening.

“Grease traps” are a common solution employed by such establishments. These are essentially boxes having one or more internal “weirs” that trap grease floating on top of passing water rather than letting it continue downstream. However, grease traps require relatively frequent and expensive maintenance: both “flushes” and also regular “pump-outs” (i.e. removal of the accumulated residue), the latter typically once a month or so.

A number of prior art solutions have been proposed to address this problem. Various chemical-based detergents are available that can be discharged into the wastewater system in an attempt to treat accumulated build-up. However, these tend to merely “saponify” the fatty residue rather than truly break it down, meaning it can reform again further downstream. In addition, the harsh chemicals involved are not only potentially harmful to the environment but also tend to damage the pipes themselves over time, increasing long-term maintenance costs.

A number of bio-friendly alternatives are also known, using agents such as bacteria (in powdered or liquid form) instead of chemicals. These agents are flushed through the wastewater system at regular intervals. However, these methods tend to be relatively ineffective as well as inefficient. They typically require regular purchase and delivery of expensive treatment ingredients to the restaurant premises. Discharging the agents into the system also requires large amounts of water.

Moreover, due to being “flushed through” at significant speed, the agents tend to have only fleeting contact with the affected areas of the system (and potentially little or no contact at all with sharp pipe bends, corners, and other hard-to-reach places—which may also be the places where residue is most prone to build up). This means the agents do not have a proper opportunity to attach to and break down the accumulated residue. This in turn means that, for such agents to have appreciable effect, they must be supplied to the wastewater system in large quantities, or even on a continuous or substantially continuous basis; and/or must be supplemented with other treatment agents.

The applicant's earlier application, WO 2016/067121, discloses technology for ameliorating the “flush-through” problem; namely by using a foaming agent to deliver a treatment substance to problematic areas. The contents of that application are incorporated herein by reference. However, while improving the efficiency and effectiveness with which the treatment substance is able to work, that system still requires regular “upstream” replenishment of the treatment ingredients, which can be relatively costly and labour-intensive.

It is accordingly an object of the present invention to provide an improved micro-organism-based wastewater system treatment method that effectively and efficiently treats food-related or other undesirable build-up in wastewater systems.

It is a further object of the invention to provide an improved method for treating an undesirable substance in an affected environment using one or more types of micro-organisms.

It is a further object of the invention to provide the public with a useful choice.

### SUMMARY OF INVENTION

According to one aspect of the invention, there is provided an onsite method of treating an undesirable substance in an affected environment using micro-organisms, the method comprising:

cultivating, in a cultivation step, one or more types of micro-organisms in a culture medium to achieve a desired or effective population of the micro-organisms; and

using a carrier to deliver the cultivated micro-organisms to the affected environment.

Preferably, the undesirable substance comprises fat, oil, grease, and/or undesired micro-organisms (and/or their by-products) such as polysaccharide slime or other biofilms.

Preferably, the affected environment comprises a component of a wastewater system; and more preferably, a grease trap, wet well, drain, pipe, or other component of a system via which food waste is disposed of.

Preferably, the micro-organisms are enzyme- and/or cellulase-producing micro-organisms; and more preferably, lypolytic enzyme-producing micro-organisms and/or lipase enzyme-producing micro-organisms.

Preferably, the micro-organisms are bacteria. More preferably, the micro-organisms are bacteria of the Bacillus genus. Even more preferably, the micro-organisms are bacteria of the Bacillus subtilis species and/or the Bacillus subtilis Complex.

Alternatively, the bacteria are selected from one or more of the group comprising: Staphylococcus aureus, Pseudomonas, Rhodococcus, Mycobacterium, Nocardia, Flavobacterium, Corynebacterium, Clostridium, Acinetobacter, Thiobacillus, Serratia, Arthrobacter, Lactobacillus, Alcanivorax, and Paenibacillus.

Preferably, the bacteria are cultivated from a dormant state, for example, by using bacteria endospores as a starter culture.

Preferably, the starter culture is provided in liquid form; for instance by combining powdered bacteria endospores with a liquid such as water to yield the starter culture in liquid form.

Preferably, the culture medium comprises ingredients suitable for initiating vegetative growth of the micro-organisms and nourishing the micro-organisms during cultivation.

Preferably, the culture medium comprises one or more nutrients selected from: beef extract, glucose/sugar, yeast, refined milk protein, soluble proteins such as peptone, corn starch, and/or bran.

More preferably, the culture medium comprises glucose, peptone and yeast.

Preferably, the culture medium is provided in liquid form; for instance by combining the one or more nutrients, in powder form, with a liquid such as water to yield the culture medium in liquid form.

Preferably, during cultivation the micro-organisms are exposed to air.

Preferably, during cultivation the micro-organisms are exposed to heat; more preferably to a temperature of between substantially 25° C. and 65° C.; and even more preferably to a temperature of substantially 35° C.

Preferably, the cultivation step continues for a predetermined period of time to achieve the desired or effective population of the micro-organisms.

Preferably, the cultivation step continues for between substantially 1 hour and substantially 6 days. More preferably, the cultivation step continues for substantially 24 hours.

Preferably, the carrier is a foaming agent.

Preferably, the foaming agent comprises one or more surfactants.

Preferably, the one or more surfactants is selected from the group comprising: anionic surfactant, cationic surfactant, zwitterionic surfactant, and/or non-ionic surfactant.

Preferably, the surfactant is a biosurfactant.

Preferably, the biosurfactant is selected from one or more of the group comprising: glycolipids; lipopeptides and lipoproteins; fatty acids, neutral lipids, and phospholipids; polymeric surfactants; and particulate biosurfactants.

Preferably, the biosurfactant is surfactin.

Preferably, one or more distinct doses of the carrier are used to deliver the cultivated micro-organisms to the affected environment.

According to another aspect of the invention, there is provided an apparatus for treating an undesirable substance in an affected environment using micro-organisms, the apparatus comprising:

at least one cultivation vessel, wherein the at least one cultivation vessel is in fluid communication with a starter micro-organism supply source, a culture medium supply source, and a carrier supply source;

wherein the at least one cultivation vessel is in fluid communication with the affected environment.

Preferably, the at least one cultivation vessel is associated with an air source. Preferably, the air source is provided by an aerator and an aeration conduit terminating inside the at least one cultivation vessel.

Preferably, the at least one cultivation vessel is associated with a heat source. Preferably, the heat source is provided by a heating element disposed in the at least one cultivation vessel.

Preferably, the apparatus comprises a plurality of cultivation vessels.

Preferably, the starter micro-organism supply source, culture medium supply source, and carrier supply source comprise, respectively, a starter micro-organism supply chamber, a culture medium supply chamber, and a carrier supply chamber.

Preferably, each of the starter micro-organism supply chamber, culture medium supply chamber, and carrier supply chamber are in fluid communication with the at least one cultivation vessel via at least one conduit.

Preferably, each conduit is associated with a pump.

Preferably, the at least one cultivation vessel comprises an outlet via which the at least one cultivation vessel is placed in fluid communication with the affected environment.

Preferably, the outlet is proximate an upper end of the at least one cultivation vessel such that, on introduction of the carrier into the at least one cultivation vessel, at least some of the cultivated micro-organisms are borne through the outlet out of the at least one cultivation vessel and delivered to the affected environment.

Preferably, the apparatus further comprises a controller configured to control the apparatus in use.

Preferably, the apparatus comprises a storage vessel in fluid communication with the at least one cultivation vessel, the storage vessel configured to store for subsequent use (for example as a biological cleaner/disinfectant) at least some of the cultivated micro-organisms.

Preferably, the at least one cultivation vessel is in fluid communication with the storage vessel via a secondary outlet on the at least one cultivation vessel; the secondary outlet preferably being proximate a lower end of the at least one cultivation vessel; the secondary outlet preferably having associated with it means for selectively allowing or preventing cultivated micro-organisms to flow from the at least one cultivation vessel to the storage vessel.

Preferably, the storage vessel has associated with it means for introducing into the storage vessel one or more substances to arrest the cultivation process so the micro-organisms can be preserved for subsequent use.

According to another aspect of the invention, there is provided a system for treating an undesirable substance in an affected environment using micro-organisms, the system comprising:

using at least one cultivation vessel which is in fluid communication with the affected environment, to cultivate one or more types of micro-organisms in a culture medium to achieve a desired or effective population of micro-organisms; and

introducing a carrier into the at least one cultivation vessel, wherein the carrier delivers the cultivated micro-organisms from the at least one cultivation vessel to the affected environment.

Preferably, the undesirable substance comprises fat, oil, grease, and/or undesired micro-organisms (and/or their by-products) such as polysaccharide slime or other biofilms.

Preferably, the affected environment comprises a component of a wastewater system; and more preferably, a grease trap, wet well, drain, pipe, or other component of system via which food waste is disposed of.

Preferably, the micro-organisms are enzyme- and/or cellulase-producing micro-organisms; and more preferably, lypolytic enzyme-producing micro-organisms and/or lipase enzyme-producing micro-organisms.

Preferably, the micro-organisms are bacteria. More preferably, the micro-organisms are bacteria of the Bacillus genus. Even more preferably, the micro-organisms are bacteria of the Bacillus subtilis species and/or the Bacillus subtilis Complex.

Alternatively, the bacteria are selected from one or more of the group comprising: Staphylococcus aureus, Pseudomonas, Rhodococcus, Mycobacterium, Nocardia, Flavobacterium, Corynebacterium, Clostridium, Acinetobacter, Thiobacillus, Serratia, Arthrobacter, Lactobacillus, Alcanivorax, and Paenibacillus.

Preferably, the bacteria are cultivated from a dormant state, for example, by using bacteria endospores as a starter culture.

Preferably, the starter culture is provided in liquid form; for instance by combining powdered bacteria endospores with a liquid such as water to yield the starter culture in liquid form.

Preferably, the culture medium comprises ingredients suitable for initiating vegetative growth of the micro-organisms and nourishing the micro-organisms during cultivation.

Preferably, the culture medium comprises one or more nutrients selected from: beef extract, glucose/sugar, yeast, refined milk protein, soluble proteins such as peptone, corn starch, and/or bran.

More preferably, the culture medium comprises glucose, peptone and yeast.

Preferably, the culture medium is provided in liquid form; for instance by combining the one or more nutrients, in powder form, with a liquid such as water to yield the culture medium in liquid form.

Preferably, the starter micro-organisms (i.e. starter culture) and culture medium are, respectively, provided to the at least one cultivation vessel from a micro-organism supply source and a culture medium supply source, the micro-organism supply source and culture medium supply source being in fluid communication with the at least one cultivation vessel.

Preferably, the micro-organism supply source and the culture medium supply source are, respectively, a micro-organism supply chamber and a culture medium supply chamber, the chambers each being in fluid communication with the at least one cultivation vessel via a conduit.

Preferably, during cultivation the micro-organisms are exposed to air.

Preferably, the at least one cultivation vessel is associated with an air source. Preferably, the air source is provided by an aerator and an aeration conduit terminating inside the at least one cultivation vessel.

Preferably, during cultivation the micro-organisms are exposed to heat; more preferably to a temperature of between substantially 25° C. and 65° C.; and even more preferably to a temperature of substantially 35° C.

Preferably, the at least one cultivation vessel is associated with a heat source. Preferably, the heat source is provided by a heating element disposed in the at least one cultivation vessel.

Preferably, cultivation continues for a predetermined period of time to achieve the desired or effective population of the micro-organisms.

Preferably, the cultivation step continues for between substantially 1 hour and substantially 6 days. More preferably, the cultivation step continues for substantially 24 hours.

Preferably, the carrier is a foaming agent.

Preferably, the foaming agent comprises one or more surfactants.

Preferably, the one or more surfactants is selected from the group comprising: anionic surfactant, cationic surfactant, zwitterionic surfactant, and/or non-ionic surfactant.

Preferably, the surfactant is a biosurfactant.

Preferably, the biosurfactant is selected from one or more of the group comprising: glycolipids; lipopeptides and lipoproteins; fatty acids, neutral lipids, and phospholipids; polymeric surfactants; and particulate biosurfactants.

Preferably, the biosurfactant is surfactin.

Preferably, the at least one cultivation vessel is in fluid communication with a carrier supply source via which the carrier is introduced to the at least one cultivation vessel.

Preferably, the carrier supply source comprises a carrier supply chamber in fluid communication with the at least one cultivation vessel via a conduit.

Preferably, the carrier is introduced into the at least one cultivation vessel in one or more distinct doses.

Preferably, the at least one cultivation vessel comprises an outlet proximate an upper end of the at least one cultivation vessel such that, on introduction of the carrier into the at least one cultivation vessel, at least some of the cultivated micro-organisms are borne through the outlet out of the at least one cultivation vessel and delivered to the affected environment.

Preferably, a plurality of cultivation vessels are used.

Preferably, the carrier is introduced into each of the plurality of cultivation vessels at different times, such that the cultivated micro-organisms from each of the plurality of cultivation vessels are delivered to the affected environment at different times, in a “staggered” fashion.

Preferably, a storage vessel is in fluid communication with the at least one cultivation vessel, the storage vessel configured to store for subsequent use at least some of the cultivated micro-organisms.

Preferably, the at least one cultivation vessel is in fluid communication with the storage vessel via a secondary outlet on the at least one cultivation vessel; the secondary outlet preferably being proximate a lower end of the at least one cultivation vessel; the secondary outlet preferably having associated with it means for selectively allowing or preventing cultivated micro-organisms to flow from the at least one cultivation vessel to the storage vessel.

### BRIEF DESCRIPTION OF DRAWINGS

Further aspects and advantages of the invention will become apparent by reference to the following Annexures, which are given by way of example only and in which:

FIG. 1 is a schematic showing a first preferred exemplary embodiment of the apparatus of the present invention; and

FIG. 2 is a schematic showing a second preferred exemplary embodiment of the apparatus of the present invention.

### DETAILED DESCRIPTION OF DRAWINGS

The following description references the invention being used to treat (i.e. break down, biodegrade, digest and/or eliminate) food-related build-up or residue (such as FOG deposits and/or biofilms such as polysaccharide slime) in wastewater systems such as grease traps, wet wells and pipes. The invention is particularly well-suited to this application. It is especially optimal that the apparatus be installed (and/or the method effected) in “upstream” portions of the wastewater system, i.e. those regions proximate the establishment discharging the food waste. In this way, the invention treats the food-related residue early on, before it is swept further downstream into the system.

However, this is not intended to be limiting and it will be understood that the invention is also suited to treating a wide variety of undesirable substances in a wide variety of affected environments. For instance, with appropriate modifications as will be readily envisaged by the skilled person, the invention may equally be applied to treating oil spills; treating oil-contaminated soils; bioremediation of sites (including water) contaminated with toxic heavy metals like uranium, cadmium, and lead; treating bio-pathogens such as E. coli in wastewater drains and other affected environments; and/or treatment of agricultural effluent in effluent ponds, such as effluent discharged from cow sheds (the micro-organisms speed up the rate at which the effluent becomes bioavailable).

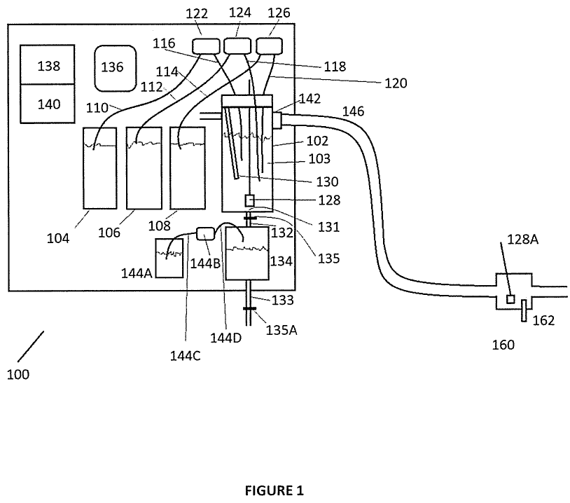

FIG. 1 is a schematic showing a first preferred exemplary embodiment of the apparatus of the present invention.

The apparatus (generally indicated by 100) comprises a cultivation vessel (102) (in this embodiment there is only a single cultivation vessel), which is located onsite, that is, at or proximate the location of the affected environment to be treated. The cultivation vessel being located onsite is optimal in that the micro-organisms are grown in situ from a relatively small volume of initial ingredients, avoiding the need to transport/deliver the cultivated micro-organism solution. Also, the cultivation vessel being onsite means the carrier, once introduced into the vessel, can readily deliver the micro-organisms to the affected environment without the need for pumps or other delivery means.

The cultivation vessel (102) is in fluid communication with a micro-organism supply source, a culture medium supply source, and a carrier supply source (collectively “the ingredient sources”). In this embodiment the ingredient sources are respectively provided by chambers 104, 106, and 108, which in this embodiment are located on the apparatus itself. While it is within the scope of the invention for the ingredient sources to be located remotely and delivered (such as by pumping) to the cultivation vessel (102), it is advantageous that they be located on or proximate the apparatus (100) as this facilitates a relatively compact, convenient and “self-sufficient” in-situ solution. The respective chambers (104, 106, 108) are in fluid communication with the cultivation vessel via conduits 110, 112, 114, 116, 118, and 120, and pumps 122, 124, and 126. The pumps may be peristaltic pumps or any other suitable kind of pump known to one skilled in the art.

It will be appreciated that the cultivation vessel (102) and the chambers (104, 106, 108) may be of any suitable capacity. For instance, the cultivation vessel (102) may range from several litres (or even less) in volume, such as when used in domestic applications, to hundreds or even thousands of litres, depending on the nature of the environment being treated. The apparatus (100) generally may be scaled up or down to any size, depending on the requirements of a given application; and it will be appreciated that not just the vessel/chambers, but also ancillary components such as the air source and heat source, may be scaled up or down as appropriate. For sake of illustration, reference is made herein to the cultivation vessel (102) and the ingredient chambers (104, 106, 108) each having a 1 L capacity; however this is not intended to be limiting.

The cultivation vessel (102) also comprises an outlet (142) proximate its upper end, the outlet (142) connected to a delivery conduit (146) in fluid communication, in use, with the affected environment in the target area (notionally indicated by 160), such as a grease trap, wet well, and/or pipe downstream of the cultivation vessel (102).

In this embodiment, the cultivation vessel (102) is associated with an air source, namely aerator (136) in fluid communication with the vessel via aeration conduit (128). The aerator (136), aeration conduit (128) and any associated componentry may be of any suitable kind, such as apparatus used to aerate fish tanks.

In this embodiment, the cultivation vessel (102) is associated with a heat source, namely heating element (130) disposed in the cultivation vessel (although it is possible for the heat source to be otherwise configured and otherwise associated with the cultivation vessel). The heating element (130) is powered, in use, by a power source (140). The power source (140) may be of any suitable type, for instance a battery, motor, or solar panel (plus battery); and may be disposed on or proximate the apparatus (100), or alternatively may be remote therefrom, and suitably connected to the apparatus (100).

The apparatus (100) also comprises a controller (138), such as a programmable logic controller (PLC), which, in use, controls the operation of the apparatus (100), such as in the manner discussed below. Operation of the apparatus (100) may be totally automated, partially automated, and/or the controller (138) may be configured to receive and execute input commands from a user. It is within the scope of the invention for the user to communicate with the controller (138) remotely, such as via an app on a smartphone or via an offsite computer.

The apparatus may optionally comprise an electronics enclosure (not shown) to house the power source (140), the controller (138), and other optional electronic components such as relays, in a watertight manner.

In this embodiment, the apparatus further comprises a storage vessel (134) in fluid communication with the cultivation vessel (102) via a conduit (132) extending between a secondary outlet (131) of the cultivation vessel (102) and the storage vessel (134); the conduit (132) having associated therewith means (135) for controlling flow, such as a tap. In this embodiment, the secondary outlet (131) is located at the base/bottom of the cultivation vessel (102); however this is not intended to be limiting.

The storage vessel (134) is configured to store for subsequent use at least some of the micro-organisms cultivated in the cultivation vessel (102) (either before or after addition of the carrier). For instance, it may be desired to use some of the cultivated micro-organisms as a biological cleaner in the manner of an ordinary detergent and/or disinfectant. Means (144A, 144B, 144C, 144D—in this case a pump mechanism similar to the others) may be provided for introducing into the storage vessel (134) one or more substances to “preserve” the micro-organisms, i.e. cause them to “hibernate” (enter dormancy) to arrest the cultivation process so the micro-organisms can be kept for later use. There are a number of known ways to achieve this, such as by the use of over-salting. A conduit (133) associated with the storage vessel (134) allows the storage vessel (134) to be drawn from; and has associated with it means (135A) for controlling flow, such as a tap. Of course, it will be appreciated that other configurations of the storage vessel and associated components are possible.

An exemplary method/system of the present invention will now be described, with reference to the apparatus of FIG. 1. The method/system will be described as being applied for treating wastewater systems; however this is not intended to be limiting.

The first step of the method comprises cultivating one or more types of micro-organisms in a culture medium for a predetermined period of time to achieve a desired or effective population of the micro-organisms.

In this embodiment, the micro-organisms are bacteria, more particularly bacteria of the Bacillus genus, and even more particularly bacteria of the Bacillus subtilis species and/or the Bacillus subtilis Complex. The skilled person will appreciate that the Bacillus subtilis Complex relates to a group of closely-related bacterial types comprising four clades:

Clade I consisting of B. subtilis including its three subspecies subtilis, spizenii, and inaquosorum, as well as B. tequilensis, B. vallismortis, B. mojavensis, and B. atrophaeus;

Clade II consisting of B. siamensis, B. amyloliquefaciens, and a conspecific complex consisting of B. methylotrophicus, B. velezensis, and B. amyloliquefaciens subsp. plantarum;

Clade III consisting of B. licheniformis and related species;

Clade IV consisting of B. pumilus and related species.

The inventor has found that it is especially advantageous to use Bacillus subtilis for treatment of wastewater systems as these bacteria have a keen appetite for the FOG build-up. FOGs are basically triglycerides, and by using bacteria, such as Bacillus subtilis, that produces lipase enzymes (fat-eating enzymes, and a type of lypolytic enzyme), the enzymes remove one of the legs off the triglycerides and free the carbon in the FOG for reproduction of the bacteria. Once the enzymes have finished with the FOG, it is no longer a triglyceride so therefore cannot reform as a solid fat.

Triglycerides are changed into diglycerides and free fatty acids (see diagram below: PL2, TL or PL1 is the enzyme in this case a Phospholipid Triglyceride Lipase enzyme).

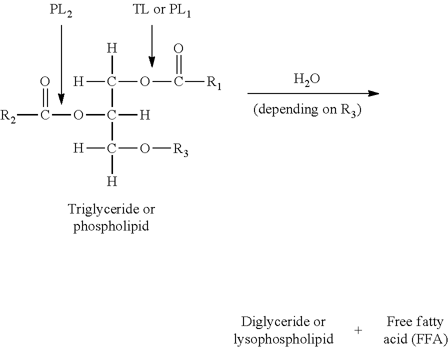

In addition to treating FOG, Bacillus subtilis effectively outcompete other, undesirable, bacteria in the wastewater system, such as sugar-related bacteria responsible for polysaccharide slime build-up, as well as other types of biofilms and bio-pathogens. They also help control unpleasant odour caused by H₂S in the wastewater system, as well as reducing damage to the system resulting from the H₂S being converted to HCl on contact with water.

For convenience, reference will henceforth be made to the micro-organisms of the invention being bacteria, and in particular Bacillus subtilis.

However, this is not intended to be limiting. Other Bacillus species may also be suitable; as may other species/genera of bacteria, such as but not limited to: Staphylococcus aureus, Pseudomonas, Rhodococcus, Mycobacterium, Nocardia, Flavobacterium, Corynebacterium, Clostridium, Acinetobacter, Thiobacillus, Serratia, Arthrobacter, Lactobacillus, Alcanivorax, and Paenibacillus; or even other types of micro-organisms altogether. The skilled person will appreciate that new types of bacteria are constantly being identified as bacteria mutate and “cross-breed”. As such, the above list is by no means intended to be limiting.

The type of micro-organism used may also vary depending on the application to which the invention is being put. For instance, when used treat agricultural effluent (such as from cow sheds) in effluent ponds, a preferable micro-organism to use may be either Lactobacillus bacteria and/or Bacillus subtilis bacteria. When used to treat oil spills, a preferable micro-organism to use may be Alcanivorax borkumensis. When used to treat oil-contaminated soils, a preferable micro-organism to use may be Alcanivorax borkumensis; or alternatively Bacillus and/or Paenibacillus. When used to treat pathogens such as E. coli, a preferable micro-organism to use may be Alcanivorax borkumensis.

It is also possible for the invention to comprise a combination of more than one type of bacteria/micro-organism, which could be specifically selected and targeted to the particular application.

The skilled person will readily arrive at bacteria/micro-organisms (one or more) suitable for use in the present invention; for instance by having regard to the type of undesirable substance that is being targeted and the type(s) of bacteria/micro-organisms that are apt to most effectively degrade/digest that undesirable substance, such as with regard to the type of enzymes the micro-organisms produce.

In this embodiment, the bacteria are cultivated from a dormant state, that is, as endospores; and the endospore starter culture is provided in liquid form, for instance by combining powdered bacteria endospores with liquid, such as water, to yield the liquid starter culture. The inventor has found that this is preferable in that a relatively small volume of liquid endospore formulation yields a relatively large quantity of usable cultivated bacteria. For instance, a substantially 1-litre supply of liquid endospore formulation can yield as many as 100 1-litre formulations of cultivated bacteria (prior to addition of surfactant).

However, this is not intended to be limiting and variations are within the scope of the invention. For instance, the bacteria/micro-organisms might not be cultivated from a dormant state; and/or they may be provided in other than a liquid form; for example, a powdered form.

Optionally, an enzyme solution (such as Sanizyme™ Boost) comprising one or more enzymes and/or one or more nutrients and/or trace elements may also be added to the liquid endospore formulation to promote and/or speed up cultivation.

The liquid endospore formulation is provided to supply chamber (104), which is in fluid communication with the cultivation vessel (102) via conduits (110) and (116). In this embodiment, the liquid endospore formulation is provided to the supply chamber (104) manually, i.e. by pre-filling of supply chamber (104) at required intervals, for instance once every three months.

It may be desirable to coordinate the amounts of liquid endospore formulation (i.e. starter micro-organism), culture medium, and carrier supply source, that are supplied to the system such that each requires replenishment at approximately the same time intervals, allowing them to all be replenished at the same time. However, the respective ingredients may equally be provided in different quantities and/or replenished individually, as and when required.

The culture medium comprises ingredients suitable for initiating vegetative growth and nourishing the bacteria during cultivation. The precise ingredients of the culture medium will depend on the type(s) of bacteria (or other micro-organism) being used, and their nutritional requirements. A range of culture medium formulations are known in the art, such as various commercially-available “nutrient broths” that may include, for instance, beef extract, glucose/sugar, yeast, refined milk protein, soluble proteins such as peptone, corn starch, and/or bran. A wide range of other ingredients may also be suitable.

The inventor has found that a particularly effective culture medium for cultivating Bacillus subtilis for the purposes of treating FOG et cetera in wastewater systems comprises a combination of yeast, peptone, and glucose. However, this may change depending on, among other things, the particular application to which the invention is being put and/or the type of bacteria being used. For instance, when Alcanivorax borkumensis are used, the optimal culture medium may comprise saline solution combined with various minerals, such as sodium, chloride, potassium, calcium, magnesium, copper, zinc among other beneficial trace elements.

In this embodiment, the culture medium is provided in liquid form, such as by combining the appropriate nutrient(s) and/or mineral(s), in powder form, with a liquid such as water to yield the culture medium in liquid form. However, this is not intended to be limiting and variations are possible, such as the culture medium being provided in powder form.

The culture medium is provided in liquid form to supply chamber (106), which is in fluid communication with the cultivation vessel (102) via conduits (112) and (118). In this embodiment, the culture medium is provided to the supply chamber (106) manually, i.e. by pre-filling of supply chamber (106) at required intervals, for instance once every three months.

In this embodiment, cultivation takes place in water, which is selectively provided (such as by means of a solenoid valve) to the cultivation vessel (102) via water conduit (150). However, this is not intended to be limiting and it is within the scope of the invention for cultivation to take place in the liquid cultivation medium itself, without the addition of water.

Optimally, the water is heated to the required temperature (see below) prior to the endospore formulation and culture medium being pumped (via pumps (122) and (124)) from the respective chambers (104) and (106) into the cultivation vessel (102). In this embodiment, substantially 10 millilitres (respectively) of the endospore formulation and culture medium are added to substantially 1 litre of water. However, the skilled person will appreciate that these quantities and ratios may differ.

In this embodiment, cultivation takes place in the presence of heat and air. The aerator (136) and aeration conduit (128) aerate the contents of the cultivation vessel (102) throughout the cultivation step; and the heating element (130) provides heat to the contents of the cultivation vessel (102) throughout the cultivation step. The inventor has found that this is conducive to cultivation, particularly where Bacillus subtilis are being used since growth is much more rapid in aerobic conditions at a higher than ambient temperature. However, this may not necessarily be the case for other micro-organisms, so the invention can be adapted accordingly in view of the particular type(s) of micro-organism being cultivated.

In this embodiment, the temperature throughout the cultivation step is maintained at between substantially 25° C. and 65° C.; and in particular at a temperature of substantially 35° C. The inventor has found that cultivation of Bacillus subtilis is optimised in this temperature range. However, other types of micro-organisms may require different cultivation temperatures.

In this embodiment, the cultivation step continues for substantially 24 hours; and more particularly for substantially 23 hours 50 minutes. This cultivation period may be optimal for several reasons. It may accord approximately with the frequency at which it is desired to discharge the bacteria to the wastewater system to effect treatment (i.e. daily). It may also correspond to the maximal possible bacterial population (experimentally, substantially 35 times the original endospore population) in view of the culture medium initially supplied—that is to say, after 24 hours nutrients may become depleted and the bacterial population in the cultivation vessel may accordingly decline.

However, this is not intended to be limiting and the invention may be adapted to allow for a different cultivation period. For instance, further culture medium may be periodically provided to the cultivation vessel (102) to replenish nutrients and permit a longer cultivation period. It is even possible for the cultivation period to be abridged, i.e. a lesser bacterial population to be attained, and the bacteria then delivered to the wastewater system where they feed off the FOG and other substances and continue to cultivate within the wastewater system itself.

Once the cultivation step is completed, the second step in the method is using a carrier to deliver the cultivated bacteria to the wastewater system.

Preferably, the carrier is a foaming agent, and reference to same will be made throughout the remainder of the specification. However, this is not intended to be limiting. It is conceivable that a different type of carrier could be used, for instance a thixotropic agent. The skilled person will appreciate suitable modifications that would be required to the apparatus of FIG. 1 in this case; for instance, pumping means may need to be associated with the cultivation vessel to urge the bacteria-infused carrier out of the vessel and deliver it to the wastewater system.

The use of a foaming agent as a carrier is advantageous for several reasons. The cultivated bacteria become “suspended” in the foam, which, once in the wastewater system, settles on and coats/clings to the various components of the system. Thus, the bacteria become “ensconced” at the site of the FOG or other undesirable substances, by attaching to and utilising the substances as substrates, and thereby breaking down the undesirable substances (preferably with the help of the surfactant comprising the foaming agent, as discussed below). This is in contrast to traditional methods which simply “flush” agents through the system at a rapid rate, meaning their contact with the undesirable substances is fleeting.

A further benefit is that the bacteria-infused foaming agent floats on top of the water once discharged into the wastewater system, which again puts the bacteria into direct and sustained contact with the FOG in the water (which likewise floats on top), enabling the bacteria to establish itself and utilise the FOG as a substrate. In addition, the floating foam is more likely than a conventional “flushed agent” to reach and coat/settle in hard-to-reach corners, bends or nooks of the wastewater system.

In this embodiment, the foaming agent comprises a surfactant (as that term will be understood by one skilled in the art, namely a substance that tends to lower surface tension). Surfactants are particularly advantageous in that they cause emulsification of the FOG, increasing its surface area so that the bacteria can in turn more effectively and rapidly digest the FOG in the manner described above. For convenience, the foaming agent will hereafter be described as being a surfactant. However, this is not intended to be limiting and it is within the scope of the invention to use a foaming agent other than a surfactant.

The type and characteristics of the surfactant are selected based on the type of FOG being treated. The skilled person will readily appreciate how to select an appropriate surfactant in this regard, so as to most effectively enable the surfactant to emulsify the FOG and increase its surface area for acting as a substrate for the bacteria. For instance, the type of FOG will inform which one or more of the following types of surfactant is appropriate: anionic surfactant, cationic surfactant, zwitterionic surfactant, and/or non-ionic surfactant. In the case of non-ionic surfactant, the type of FOG will also determine the required Hydrophilic-Lipophilic Balance (HLB) of the surfactant.

The skilled person will also appreciate that more than one surfactant can be combined to obtain a blended surfactant having the required characteristics. An appropriate surfactant can come from any family of surfactants and can be blended to achieve the best outcome for the specific environment and requirements. For example McCutcheon's Emulsifiers & Detergents, International Edition, 1998 lists a number of suitable examples at pages 223 to 231 of the HLB index.

It may be particularly effective for the surfactant to be a biosurfactant. Biosurfactants are surface-active molecules synthesized by micro-organisms. They are known to have broad-range functional properties; most notably they interfere with/destroy the structure of undesirable substances such as bacteria or biofilms. Compared with chemical surfactants, they are selective, required in small quantities, effective under broad ranges of conditions (including being effective at promoting treatment of oil-spills and contaminated soil, as well as other types of contamination), and environmentally friendly. Thus an embodiment of the invention using a biosurfactant would work in a synergistic, or “two-pronged” way. In addition to acting as a carrier to deliver the micro-organisms to the site of the undesirable substances, the biosurfactant would biodegrade the undesirable substances in its own right.

A range of biosurfactant types are known and could be employed in the present invention. These include: glycolipids; lipopeptides and lipoproteins; fatty acids, neutral lipids, and phospholipids; polymeric surfactants; and particulate biosurfactants.

When treating wastewater systems, it may be especially effective to use surfactin specifically. Surfactin is a very powerful surfactant produced by Bacillus subtilis bacteria. Surfactin is capable of penetrating the cell membranes of other types of bacteria by creating a permeable environment for the lipid bilayer of the bacterial membrane and causing disruption that solubilizes the membrane. Thus, an embodiment of the invention using Bacillus subtilis as the micro-organism and surfactin as the surfactant would work in a synergistic, or “two-pronged” way. In addition to acting as a carrier for the Bacillus subtilis bacteria, the surfactin would, once in contact with the undesirable substances in the wastewater system, biodegrade these in its own right, alongside the Bacillus subtilis.

When used for the treatment of oil spills, the biosurfactant may for instance be of a kind produced by the P. aeruginosa bacteria (such as a glycolipid, and more particularly a rhamnolipid); of a kind produced by the Arthrobacter strain EK1 bacteria or the Arthrobacter strain S-II bacteria; of a kind produced by the Alcaligenes strain MM-1 bacteria; and/or of the kind produced by cyanobacteria. When used for the treatment of oil-contaminated soils, the biosurfactant may be of a kind produced by the P. aeruginosa bacteria, such as a glycolipid.

Referring again to FIG. 1, once the cultivation step has been completed, the surfactant is pumped into the cultivation vessel (102) from supply chamber (108) via pump (126) and conduits (114, 120). In this embodiment, the surfactant has been provided to the supply chamber (108) manually, i.e. by pre-filling of supply chamber (108) at regular intervals, for instance once every three months.

On contact with the liquid (103) in the cultivation vessel (102), the surfactant foams up. The cultivated bacteria (103) in the cultivation vessel (102) become “suspended” in the surfactant. The bacteria-infused surfactant bubbles up and overflows out of the outlet (142) and down the delivery conduit (146), which is oriented such that the surfactant is advantageously discharged into the wastewater system under gravity (although other configurations may be possible). Once in the system, it floats on the water surface, coating/clinging to exposed portions of the wastewater system (such as the inside of pipes, and/or walls of grease traps/wet wells) as well as directly contacting the FOG layer also floating on the water surface. Sustained contact of the bacteria with the FOG and other undesirable substances (such as polysaccharide slime and other biofilms) is thereby achieved, allowing the bacteria to utilise the substances as substrates and digest, biodegrade, and/or outcompete these substances.

In this embodiment, the surfactant is introduced in several distinct doses; and more particularly in 3 distinct doses over the space of substantially 10 minutes, each of which causes approximately one-third of the cultivated bacteria (103) in the cultivation vessel (102) to be discharged to the wastewater system. Discharging in smaller doses optimises the effectiveness of the system.

Optionally, the process may include diverting some part of the cultivated bacteria to the storage vessel (134) for later use, as described above. This may occur either before or after introduction of the surfactant.

In this embodiment, after the bacteria have been discharged, the apparatus (100) is rinsed, and may also be sterilised/disinfected to eliminate any residual substances prior to the next cultivation cycle commencing.

Variations on the above-described exemplary method are possible. For instance, in some cases it may be undesirable for the process to take place continuously across the 24-hour cycle described above. That is to say, it may be preferable for the process to be suspended/paused at some intermediate point, and then resumed; such as where a restaurant is open/staffed only part-time and operation of the system needs to be timed accordingly. In such a case, the method may include, for instance, introducing into the cultivation vessel (102) one or more substances to “preserve” the micro-organisms and arrest the cultivation process. There are a number of known ways to achieve this, such as by the use of over-salting.

As noted above, in some cases it may also be desired to discharge the bacteria into the wastewater system earlier, that is to say, at some intermediate stage of the cultivation process; with the bacteria then using the FOG in the wastewater system as nutrients and thereby continuing to cultivate. In this regard, it may be desired to have a further aeration conduit (128A, fed by an aerator (not shown)) in the target area (160) of the wastewater system itself (such as in the grease trap or wet well), to provide air to the bacteria so cultivation may continue in the wastewater system. An aeration conduit/aerator in the target area (160) may also be advantageous even if the bacteria are fully cultivated in the cultivation vessel (102) prior to being discharged into the wastewater system.

It may also be desirable for the system to include sensors in various locations within the apparatus and/or in the target area of the wastewater system, to monitor (and/or correct or adjust) pH or other parameters that are important to the system. For instance, a pH-monitoring sensor (162) may be installed in the target area (160), to monitor the pH of the affected environment and thereby monitor the effectiveness of the system and make any required changes to the delivery of the micro-organisms to the target area. The further aeration conduit/aerator and sensor(s) may be communicative with the controller (138).

It will be appreciated that the controller (138) of the apparatus may be configured to effect some or all of the above-discussed process, whether automatically and/or by receiving and executing input from a user (including in the form of an override); wherein the user may communicate with the controller (138) remotely, such as via a smartphone app or from and offsite computer.

For instance, in a predominantly automated embodiment, assuming the supply chambers (104, 106, 108) have been pre-filled, as has the cultivation vessel (102) with water (if used in the particular embodiment): the controller (138) may, firstly, detect the appropriate/predetermined time to commence the process; may then cause delivery of the starter micro-organisms and culture medium from the respective supply chambers (104, 106) to the cultivation vessel (102) via the respective conduits (110, 112, 116, 118) by actuating the respective pumps (122, 124); may actuate the heating element (130) and aerator (136); may monitor the duration of the cultivation period; and at the end of the cultivation period may actuate pump (126) to cause the foaming agent to be delivered, optionally in several distinct doses, from supply chamber (108) to the cultivation vessel (102) via conduits (114, 120). In addition, the controller (138) may comprise a memory (not shown) that records data relating to the process for future reference.

In other embodiments, some or all of these steps may be undertaken manually and/or may be subject to an override. For instance, an operator may have discretion over whether and when to terminate/abridge the cultivation stage and proceed to the foaming/delivery stage. In another example, the operator may be able to control the quantities of ingredients being delivered to the cultivation vessel, to e.g. moderate cultivation rate, deliver additional culture medium if required, or moderate the amount and properties of the foam.

The inventors have found that, using the exemplary quantities discussed above, an efficient and cost-effective treatment system and method is achieved. Specifically:
A single supply of liquid endospore formulation (at substantially 1 litre) can yield as many as 100 1-litre formulations of cultivated bacteria (prior to addition of surfactant); wherein, in each formulation of cultivated bacteria, the bacterial population can be around 35 times that of the endospores originally introduced.
A single supply of liquid endospore formulation, culture medium and surfactant (at substantially 1 litre of each) is sufficient for around three months' worth of treatment; that is to say, the supply chambers require re-filling only once every three months;
Grease-trap maintenance (pump-outs) may be reduced from around once per month to as seldom as once per year.

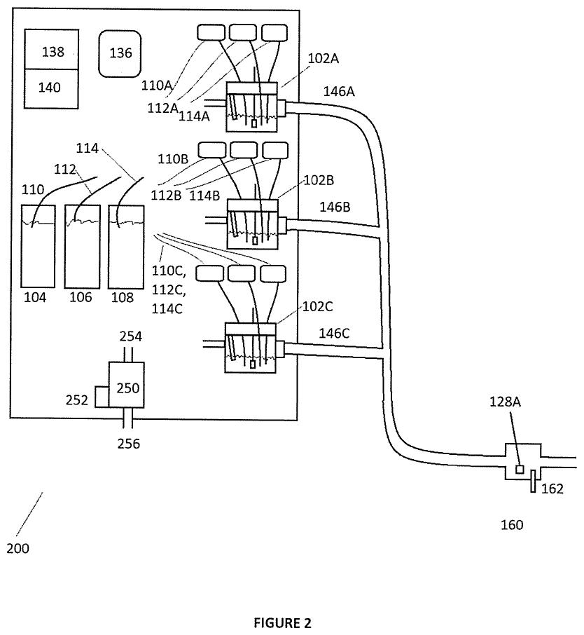

FIG. 2 is a schematic showing an apparatus (generally indicated by 200) according to a second preferred embodiment of the invention. The apparatus is similar in many respects to that of FIG. 1. For ease of presentation, the intermediate portions of the various conduits have been omitted from the drawing; however, they are coupled as indicated by the reference numerals.

There are however a number of differences. Firstly, the apparatus (200) comprises three cultivation vessels (202A, 202B, 202C). The presence of multiple cultivation vessels allows delivery of the bacteria-infused surfactant to the wastewater system to be “staggered” or occur in a “relay”. This may in turn allow more frequent treatment, since cultivation can be occurring in one cultivation vessel contemporaneously with delivery from another cultivation vessel. For instance, treatment may occur on a thrice-daily basis if desired.

Each cultivation vessel (202A, 202B, 202C) is depicted as having its own set of pumps and conduits via which it is in fluid communication with the source ingredient chambers. However, it will be appreciated that the apparatus (200) may alternatively be configured with a pump manifold, such that a single set of pumps selectively services each of the cultivation vessels (202A, 202B, 202C).

The apparatus (200) is also depicted as having a single storage vessel (schematically indicated at (250)) which is substantially the same as that in FIG. 1 and is configured to be in fluid communication with the cultivation vessels (202A, 202B, 202C); the storage vessel (250) having associated therewith means (schematically indicated at (252)) for introducing one or more substances to “preserve” the micro-organisms. In an alternative configuration, each cultivation vessel (202A, 202B, 202C) may be associated with its own storage vessel.

It will accordingly be appreciated that the present invention provides a number of advantages over the prior art.

Firstly, the invention provides a method and system for treating wastewater systems that is more effective than conventional “flush-based” methods.
The use of a foaming agent as a carrier means the micro-organisms are delivered to, and cling to, exposed surfaces of the system (including “hard-to-reach” regions) as well as floating on the water itself, and therefore also on the FOG layer atop the water. This means the micro-organisms are able to establish sustained contact with the undesirable substances in the wastewater system (meaning much more opportunity to break down those substances), which is a key advantage over “flush-based” methods where any contact is fleeting.
Where a surfactant is used as the carrier, the surfactant also emulsifies the FOG, increasing the surface area of the FOG and allowing the bacteria to digest it even more effectively, as opposed to “saponifying” it as conventional detergents tend to do.
Bacteria, particularly Bacillus subtilis, are also effective at outcompeting those undesirable bacteria responsible for biofilms such as polysaccharide slime.

At the same time, the invention is more cost-efficient than conventional methods.
A relatively small amount of “starter” ingredients is required, compared to the number of cultivated bacteria (and the volume of bacteria-infused formulation) ultimately produced;
The cultivation taking place “in situ” means reduced delivery costs (of ingredients), limited to the starter ingredients, as well as reduced packaging;
Since the foaming agent allows the bacteria to be more effective, it follows that less bacteria needs to be discharged into the drain to achieve the same effect, meaning greater cost-efficiency;
Since the method does not rely on “flushing”, it also uses significantly less water;
Due to the increased effectiveness of the method, maintenance costs (“pump-outs” and “flushes” of the grease traps and related components) are reduced;
Maintenance costs are also reduced due to less damage being done to the wastewater system over time.

Other benefits include:
Reducing “drain fly” problems by effectively treating their “breeding ground”, namely decomposing food matter;
Reducing unpleasant odour due to H₂S;
Reducing damage to pipes as a result of HCl (converted from H₂S);
Providing a wastewater treatment solution that is relatively environmentally-friendly compared to conventional solutions;
Providing a wastewater treatment solution that facilitates compliance with local government rules and regulations.

It will be understood that the above Figures and description of same have been given by way of example only, and are not intended to limit the scope of the invention. The skilled person will readily envisage variations falling within the scope of the invention.

The invention may also be said to broadly consist in the individual parts, components and features described herein, and/or any combination of same.

Throughout the present specification, the term “comprise” and related terms will be understood inclusively, that is to say, will be taken to mean an inclusion of not only the listed components directly referenced but also other non-specified components or elements.

1. An onsite method of treating an undesirable substance in an affected environment using micro-organisms, the method comprising:
cultivating, in an onsite cultivation step, one or more types of micro-organisms in a culture medium to achieve a desired or effective population of the micro-organisms; and
using a carrier to deliver the cultivated micro-organisms to the affected environment, wherein the carrier is a biosurfactant selected from one or more of the group comprising: glycolipids; lipopeptides and lipoproteins; fatty acids, neutral lipids, and phospholipids; polymeric surfactants; and particulate biosurfactants; and wherein the biosurfactant is at least partly comprised of surfactin;
wherein, in use, the biosurfactant works synergistically with the cultivated micro-organisms, wherein after delivering the cultivated micro-organisms to the affected environment, the biosurfactant works alongside the cultivated micro-organisms to biodegrade the undesirable substance in the affected environment.

2. The method of claim 1, wherein the micro-organisms are enzyme-producing micro-organisms and/or cellulase-producing micro-organisms.

3. The method of claim 1, wherein the micro-organisms are Bacillus subtilis bacteria.

4. The method of claim 1, wherein the micro-organisms are cultivated from a dormant state, said micro-organisms in said dormant state being provided as a starter culture or starter micro-organism supply provided in a liquid form.

5. The method of claim 1, wherein the culture medium is provided in a liquid form and comprises one or more nutrients selected from: beef extract, glucose/sugar, yeast, refined milk protein, soluble proteins such as peptone, corn starch, and/or bran.

6. The method of claim 1, wherein during cultivation the micro-organisms are exposed to air, and to a temperature of substantially 35° C.

7. The method of any of claim 1, wherein the cultivation step continues for substantially 24 hours.

8. The method of claim 1, wherein one or more distinct doses of the carrier are used to deliver the cultivated micro-organisms to the affected environment.

9. The method of claim 1, wherein the undesirable substance comprises one or more of: fat, oil, grease (FOG); and/or undesired micro-organisms or their by-products such as polysaccharide slime or other biofilms.

10. An apparatus for onsite treatment of an undesirable substance in an affected environment using micro-organisms, the apparatus comprising:
at least one onsite cultivation vessel, wherein the at least one cultivation vessel is in fluid communication with a starter micro-organism supply source, a culture medium supply source, and a carrier supply source for a carrier, said carrier being a biosurfactant selected from one or more of the group comprising: glycolipids; lipopeptides and lipoproteins; fatty acids, neutral lipids, and phospholipids; polymeric surfactants; and particulate biosurfactants; said biosurfactant being at least partly comprised of surfactin;
wherein the at least one cultivation vessel is in fluid communication with the affected environment, wherein the at least one cultivation vessel comprises an outlet proximate an upper end thereof such that, on introduction of the carrier into the at least one cultivation vessel, at least some micro-organisms cultivated in the at least one cultivation vessel are borne through the outlet and delivered to the affected environment.

11. The apparatus of claim 10, wherein the at least one cultivation vessel is associated with an air source and a heat source.

12. The apparatus of claim 10, wherein the apparatus comprises a storage vessel in fluid communication with the at least one cultivation vessel via a secondary outlet on the at least one cultivation vessel, the storage vessel being configured to store for subsequent use at least some of the micro-organisms, wherein the storage vessel has associated with it means for introducing into the storage vessel one or more substances to arrest the cultivation process.

13. The apparatus of claim 10, wherein the apparatus comprises a plurality of cultivation vessels.

14. The apparatus of claim 10, wherein the apparatus comprises a controller configured to control the apparatus in use.

15. A system for treating an undesirable substance in an affected environment using micro-organisms by effecting the method of claim 1 using the apparatus of claim 10, the system comprising:
in the at least one cultivation vessel, cultivating, in the cultivation step, one or more types of micro-organisms in the culture medium to achieve a desired or effective population of the micro-organisms; and
introducing the carrier into the at least one cultivation vessel to deliver at least some of the cultivated micro-organisms from the at least one cultivation vessel to the affected environment.

16. The system of claim 15, wherein the carrier is introduced into the at least one cultivation vessel in one or more distinct doses.

17. The system of claim 15, wherein there are a plurality of cultivation vessels and the carrier is introduced into each of the plurality of cultivation vessels at different times, such that the cultivated micro-organisms from each of the plurality of cultivation vessels are delivered to the affected environment at different times, in a “staggered” fashion.
